(19) United States
(12) Patent Application Publication
(10) Pub. No.: US 2005/0282153 A1
(43) Pub. Date: Dec. 22, 2005

(54) Quantification of microorganism

(76) Inventor: Yu-Chan Chao, Taipei (TW)

Correspondence Address: FISH & RICHARDSON PC, P.O. BOX 1022, MINNEAPOLIS, MN, 55440-1022, US

(21) Appl. No.: 10/873,325

(22) Filed: Jun. 21, 2004

(51) Int. Cl. C12Q 1/70; C12Q 1/68; C07H 21/04

(52) U.S. Cl. 435/5; 536/24.1; 435/6

(57) ABSTRACT

A method for quantifying a microorganism and related primers and nucleic acids.

### BACKGROUND

Titer determination, i.e., quantification, of a microorganism (e.g., a bacterium or a virus), is a prerequisite for studying and using the microorganism. For example, baculoviruses, a group of insect-borne viruses, have been successfully used for generating active engineered protein in some mammalian cells. See, e.g., King et al., The Baculovirus Expression System: A Laboratory Guide; Chapman and Hall: London, 1992; Luckow, Curr. Opin. Biotechnol. 1993, 4, 564-572; O'reilly, et al., Baculovirus Expression Vectors: A Laboratory Manual, Oxford University Press: New York, 1994; and Smith et al., 1983, Mol. Cell Biol. 3, 2156-2165. The titer of a baculovirus must be determined so as to achieve optimal infection of a host cell culture and thereby to maximize protein yield. Conventional methods for quantifying a baculovirus are tedious, time consuming, and prone to error. Take the most frequently used end-point dilution method and plaque formation method for example. Each of these two methods requires serially diluting a virus-containing sample to be quantified, seeding the diluted samples onto culturing plates, and growing the virus. The entire process typically takes 4-7 days. Thus, there is a need for a simple, rapid, and accurate quantification method.

### SUMMARY

This invention relates a simple, rapid, and accurate method for quantifying a microorganism.

In one aspect, the invention features a method for quantifying the titer of a microorganism such as a virus, e.g., a baculovirus, in a sample. The method includes (i) amplifying, by a polymerase chain reaction (PCR), a nucleic acid in the genome of the microorganism to form a double-stranded nucleic acid product; (ii) contacting the nucleic acid product with a label, such as a fluorophore, e.g., SYBR Green I, in a solution for binding therebetween; and (iii) quantifying the microorganism by monitoring a signal produced by the bound label, the intensity of which is a function of the quantity of the microorganism in the sample. The microorganism can be quantified based on the function of Formula I or II as described in the Detailed Description section and actual example below. In one embodiment, the method further includes, after the contacting step, heating the solution to a temperature that is above the melting temperature of the double-stranded nucleic acid product, and confirming the identity of the microorganism by monitoring a change in the intensity of the signal produced by the label as the temperature rises.

The just-described method can be used to quantify a baculovirus. For this purpose, one can amplify a region selected from the ie-1 gene of the baculovirus (SEQ ID NO: 3, GenBank Accession No. M21884) listed below:

(SEQ ID NO: 3)

| | |
|---|---|
| 1 | atcgatgtcc ttgtgatgcg cgcgacattt ttgtaggtta |
| | ttgataaaat gaacggatac |
| 61 | gttgcccgac attatcatta aatccttggc gtagaatttg |
| | tcgggtccat tgtccgtgtg |
| 121 | cgctagcatg cccgtaacgg acctcgtact tttggcttca |
| | aaggttttgc gcacagacaa |
| 181 | aatgtgccac acttgcagct ctgcatgtgt gcgcgttacc |
| | acaaatccca acggcgcagt |
| 241 | gtacttgttg tatgcaaata aatctcgata aaggcgcggc |
| | gcgcgaatgc agctgatcac |
| 301 | gtacgctcct cgtgttccgt tcaaggacgg tgttatcgac |
| | ctcagattaa tgtttatcgg |
| 361 | ccgactgttt tcgtatccgc tcaccaaacg cgtttttgca |
| | ttaacattgt atgtcggcgg |
| 421 | atgttctata tctaatttga ataaataaac gataaccgcg |
| | ttggttttag agggcataat |
| 481 | aaaagaaata ttgttatcgt gttcgccatt agggcagtat |
| | aaattgacgt tcatgttgga |
| 541 | tattgtttca gttgcaagtt gacactggcg gcgacaagat |
| | cgtgaacaac caagtgacta |
| 601 | tgacgcaaat taattttaac gcgtcgtaca ccagcgcttc |
| | gacgccgtcc cgagcgtcgt |
| 661 | tcgacaacag ctattcagag ttttgtgata aacaacccaa |
| | cgactattta agttattata |
| 721 | accatoccac cccggatgga gccgacacgg tgatatctga |
| | cagcgagact gcggcagctt |
| 781 | caaacttttt ggcaagcgtc aactcgttaa ctgataatga |
| | tttagtggaa tgtttgctca |
| 841 | agaccactga taatctcgaa gaagcagtta gttctgctta |
| | ttattcggaa tcccttgagc |
| 901 | agcctgttgt ggagcaacca tcgcccagtt ctgcttatca |
| | tgcggaatct tttgagcatt |
| 961 | ctgctggtgt gaaccaacca tcggcaactg gaactaaacg |
| | gaagctggac gaatacttgg |
| 1021 | acaattcaca aggtgtggtg ggccagttta acaaaattaa |
| | attgaggcct aaatacaaga |
| 1081 | aaagcacaat tcaaagctgt gcaacccttg aacagacaat |
| | taatcacaac acgaacattt |
| 1141 | gcacggtcgc ttcaactcaa gaaattacgc attattttac |
| | taatgatttt gcgccgtatt |
| 1201 | taatgcgttt cgacgacaac gactacaatt ccaacaggtt |
| | ctccgaccat atgtccgaaa |
| 1261 | ctggttatta catgtttgtg gttaaaaaaa gtgaagtgaa |
| | gccgtttgaa attatatttg |
| 1321 | ccaagtacgt gagcaatgtg gtttacgaat atacaaacaa |
| | ttattacatg gtagataatc |
| 1381 | gcgtgtttgt ggtaactttt gataaaatta ggtttatgat |
| | ttcgtacaat ttggttaaag |
| 1441 | aaaccggcat agaaattcct cattctcaag atgtgtgcaa |
| | cggcgagacg gctgcacaaa |
| 1501 | attgtaaaaa atgccatttc gtcgatgtgc accacacgtt |
| | taaagctgct ctgacttcat |
| 1561 | attttaattt agatatgtat tacgcgcaaa ccacatttgt |
| | gactttgtta caatcgttgg |
| 1621 | gcgaaagaaa atgtgggttt cttttgagca agttgtacga |
| | aatgtatcaa gataaaaatt |
| 1681 | tatttacttt gcctattatg cttagtcgta aagagagtaa |
| | tgaaattgag actgcatcta |
| 1741 | ataatttctt tgtatcgccg tatgtgagtc aaatattaaa |
| | gtattcggaa agtgtgcagt |
| 1801 | ttcccgacaa tcccccaaac aaatatgtgg tggacaattt |
| | aaatttaatt gttaacaaaa |
| 1861 | aaagtacgct cacgtacaaa tacagcagcg tcgctaatct |
| | tttgtttaat aattataaat |
| 1921 | atcatgacaa tattgcgagt aataataacg cagaaaattt |
| | aaaaaaggtt aagaaggagg |
| 1981 | acggcagcat gcacattgtc gaacagtatt tgactcagaa |
| | tgtagataat gtaaagggtc |
| 2041 | acaattttat agtattgtct ttcaaaaacg aggagcgatt |
| | gactatagct aagaaaaaca |
| 2101 | aagagtttta ttggatttct ggcgaaatta aagatgtaga |
| | cgttagtcaa gtaattcaaa |
| 2161 | aatataatag atttaagcat cacatgtttg taatcggtaa |
| | agtgaaccga agagagagca |
| 2221 | ctacattgca caataatttg ttaaaattgt tagctttaat |
| | attacagggt ctggttccgt |
| 2281 | tgtccgacgc tataacgttt gcggaacaaa aactaaattg |
| | taaatataaa aaattcgaat |
| 2341 | ttaattaatt atacatatat tttgaattta attaattata |
| | catatatttt atattatttt |
| 2401 | tgtcttttat tatcgagggg ccgttgttgg tgtggggttt |
| | tgcatagaaa taacaatggg |
| 2461 | agttggcgac gttgctgcgc caacaccacc tcctcctcct |
| | cctttcatca tgtatctgta |
| 2521 | gataaaataa aatattaaac ctaaaaacaa gaccgcgcct |
| | atcaacaaaa tgataggcat |
| 2581 | taacttgccg cgacgctgtc actaacgttg gacgatttgc |
| | cgactaaacc ttcatcgccc |
| 2641 | agtaaccaat ctaga |

using a pair of primers containing, e.g., CCCGTAACGGACCTCGTACTT (Ie-1I forward primer, SEQ ID NO: 1) and TTATCGAGATTTATTTGCATACAACAAG (Ie-1I reverse primer, SEQ ID NO: 2), respectively. One can also use the method to quantify a recombinant baculovirus that contains a heterologous nucleic acid encoding a heterologous protein, such as green fluorescent protein (GFP), luciferase, or its fragment. In this case, one can amplify a region selected from (i) the sequence encoding GFP using a pair of primers containing, e.g., CAACAGCCACAACGTCTATATCATG (SEQ ID NO: 4) and ATGTTGTGGCGGATCTTGAAG (SEQ ID NO: 5), respectively, or (ii) the sequence encoding luciferase using a pair of primers containing, e.g., CGGCGCCATTCTATCCTCTA (SEQ ID NO: 6) and ACCAGGGCGTATCTCTTCATAGC (SEQ BD NO: 7), respectively. Each of the above-mentioned primer is 5-100 (e.g., 10-50) nucleotides in length.

In another aspect, the invention features a pair of primers for amplifying a nucleic acid of baculovirus. Each primer contains an oligo-nucleotide that is selected from the baculovirus ie-1gene region and is 5-100 (e.g., 10-50) nucleotides in length. Examples of the oligo-nucleotide include SEQ ID NO: 1 and 2 mentioned above. The invention also features a nucleic acid that is obtained from amplification of a baculovirus nucleic acid template with such a pair of primers. In one example, this nucleic acid contains CCCGTAACGGACCTCGTACTTTTGGCTTCAAAGGTTTTGCGCACAGACAAAAT GTGCCACACTTGCAGCTCTGCATGTGTGCGCGTTACCACAAATCCCAACGGC GCAGTGTACTTGTTGTATGCAAATAAATCTCGATAA (SEQ ID NO: 3) or its complementary sequence. The nucleic acid is 50-2,000 (e.g., 100-1,000) nucleotides in length. Such primers and nucleic acids are used to quantify a baculovirus by the method of this invention.

The details of one or more embodiments of the invention are set forth in the accompanying description below. Other advantages, features, and objects of the invention will be apparent from the detailed description and the claims.

### DETAILED DESCRIPTION

The present invention relates to a method for quantifying a microorganism, such as a baculovirus, in a sample. This method requires using a pair of primers to amplify a target nucleic acid in the genome of the baculovirus to form a double-stranded nucleic acid product and determining the concentration of the target nucleic acid in the sample. Based on this concentration, one can quantify baculovirus in the sample.

In general, a target nucleic acid can be selected from any region of the genome of a baculovirus to be quantified. Since, for expressing an engineered protein, one often replaces certain unessential regions of the baculovirus genome with a heterologous sequence, such as a sequence encoding an engineered protein or selection marker protein (e.g., GFP), it is preferred to select a target nucleic acid from a gene or region that is essential for a baculovirus. Under certain circumstances, one can select a target nucleic acid from one of the just mentioned heterologous sequences.

After selecting a target region, one can use quantitative PCR to amplify it and quantify the amplification products. A pair of primers used for the PCR can be designed based on principles and techniques known in the art. For example, one can use software programs to select primer sequences according to the properties of the target region, e.g., GC-content, annealing temperature, or internal pairing.

PCR amplification can be carried out following standard procedures. See, e.g., Innis et al. (1990) PCR Protocols: A Guide to Methods and Applications Academic Press, Harcourt Brace Javanovich, New York. The 3 steps in PCR amplification denaturing, annealing and elongating, can be repeated as many times as needed to produce the desired quantity of an amplification product corresponding to the target nucleic acid. The required cycling number depends on, among others, the nature of the sample. If the sample is a complex mixture of nucleic acids, more cycling steps will be required to amplify the target sequence sufficiently for detection. Generally, the cycling steps are repeated at least about 10 times, but may be repeated as many as 20, 30, 40, 50, 60, or even 100 times.

To quantify a target nucleic acid in a sample, one contacts the products amplified from the nucleic acid with a label in a solution. The label can be a fluorophore, e.g., SYBR Green I, that specifically binds to double-strand DNA. In its unbound state, the fluorophores, upon irradiation by an excitation light, emits little fluorescence signal. In contrast, when bound to double-strand DNA, it emits a much stronger fluorescence signal. The fluorescence signal intensity is proportional to the amount of double-stranded DNA products generated during the PCR amplification, and, in turn, is a function of the quantity of the target nucleic acid, as well as the titer of the baculovirus, in the sample. Such a fluorophore allows one to quantify double stranded DNA PCR products as well as the baculovirus without using any nucleic acid probe specific to the baculovirus.

The above-described PCR amplification, incubation with a fluorophore, and quantification of a target nuclei acid can be conducted sequentially or simultaneously. When conducted simultaneously, quantitative real-time PCR (Q-PCR) amplification can be carried out using a commercially available Real-PCR system (e.g., LightCycler marketed by Roche Molecular Diagnostic.). To quantify a target nucleic acid, fluorescence emitted from the fluorophore is monitored at the end of each PCR cycle upon irradiation. The intensity of the fluorescence emission is a function of the amount of the amplified nuclei acid product, which, in turn, is a function of the original concentration of the target nucleic acid and PCR cycle numbers. When enough cycles are carried out, the rate of the accumulation of the amplified nuclei acid product and the rate of the change in fluorescence emission enter a log-linear phase. The PCR cycle number corresponding to the entry point (i.e., the cross point value, or Cp value) can be determined by plotting the fluorescence emission intensity against the PCR cycling number.

The Cp value thus obtained is then compared to a predetermined Cp value that corresponds to a known original concentration of a standard nucleic acid or known original titer of a baculovirus. A series of such predetermined Cp values can be obtained in the manner described below in the Example section. Accordingly, one can derive the corresponding original concentration of the target nucleic acid or original titer of the baculovirus by comparing a given Cp value to a series of predetermined Cp values.

Alternatively, one can use Formulae I or II to derive the original baculovirus titer:
y=−1.2632×Ln(x)+32.83 (Formula I)
y=−1.4293×Ln(x)+38.965 (Formula II)

In both formulae, “y” stands for a Cp (cycle number) and “x” stands for a baculovirus titer value (pfu/ml). More specifically, one can substitute a Cp value obtained in the manner described above for “y” in either of the two formulae and solve the equation for “x,” i.e., the original baculovirus titer.

Preferably, one should confirm the identity of the amplification product before quantifying a target nucleic acid. To confirm the identity, one can subject the products to a melting curve analysis at the end of the PCR amplification. The reaction is heated slowly, e.g., at a transition rate of 0.1-0.5° C./second, to a temperature higher than the expected melting temperature (Tm) of the product amplified from the target nucleic acid. Meanwhile, fluorescence emitted from the fluorophore is monitored upon irradiation by an excitation light. The intensities (F) are plotted against temperature (T) to generate a melting curve. Then, a first derivative of the melting curve (i.e., a first derivative melting curve) is generated by plotting the negative derivative of F with respect to temperature (−dF/dT) against T to locate a melting peak. The temperature value corresponding to the melting peak is then compared with the expected melting temperature. In a preferred embodiment, the melting curve analysis is performed using LightCycler analysis software 3.5 (Roche Diagnostics Applied Science, Mannheim Germany). The products are confirmed to be amplified from the target region if the temperature value is the same as the expected melting temperature.

As the method of this invention is based on quantification of microbial DNA as a proxy for the microorganism titer, care should be taken to avoid damaging the microorganism. For example, to quantify a baculovirus, one should avoid detergent treatment or repeat freezing and thawing a baculovirus sample. Also, the sample should be properly stored. As shown in Example 4 below, baculoviruses could be stored at 4° C. for at least 4 years.

When quantifying a baculovirus, the method of this invention is superior to the conventional titer determining methods, such as the end-point dilution method and the plaque formation method. Since it does not need tedious and error-prone prosecutes such as virus dilution or seeding and infecting host cells, it is simpler, more rapid, and more accurate than the conventional methods. In deed, as shown in the examples below, the method of this invention reduces the titer determination time from 5 days (by end-point dilution) to just 1 hour. Also, this method can be used over a wide viral titer range (at least between 10³ and 10⁹ pfu/mL). The method is also useful for titer determinations of large numbers of unknown virus clones, e.g., the production of hundreds of proteins for a genome project or the screening of virus clones from a baculovirus library. Since standard curves or formulae for various baculoviruses do not vary significantly, one can use the standard curve or formulae shown in the Examples below for quantifying various baculoviruses. Of course, one can also establish a standard curve or formula on his or her own in the manner described in the Examples.

The method of this invention can utilize Q-PCR technology, which has been used in determining titers of other viruses, such as adenoviruses, adeno-associated viruses, herpes simplex virus, and retroviruses. However, all of these titer determination assays adapt the TagMan probe technology (Nitsche et al., Clin. Chem. 1999, 45, 1932-1937 and Liviak et al., PCR Method Appl. 1995, 4, 357-362.). This technology uses fluorescent-labeled nucleic acid probes, such as TagMan probes, which are expensive and introduce transitional complexity to both the design and the parameters of the amplification reactions. The method of this invention does not use such nucleic acid probes. Instead, it can use a fluorophore, e.g., SYBR Green I, that specifically binds to double-strand DNA. As this fluorophore binds to the entire amplification products rather than a small region completementary to a TagMan probe, more fluorophores are associated with the amplification products. Thus, the method of this invention is simpler, less expensive, and more sensitive than the TagMan method.

The specific examples below are to be construed as merely illustrative, and not limitative of the remainder of the disclosure in any way whatsoever. Without further elaboration, it is believed that one skilled in the art can, based on the description herein, utilize the present invention to its fullest extent. All publications cited herein are hereby incorporated by reference in their entirety.

The general materials and methods for Examples 1-4 are described first:

Construction of Plasmids and Recombinant AcMNPV

Three plasmids pAPcmE, pAPhE, and pABpE were constructed for making recombinant viruses. Each plasmid contained an enhanced GFP (EGFP, Clontech, Palo Alto, Calif.) gene, driven by a heat shock 70 promoter, a minimal CMV (CMVm) promoter, or a polyhedrin promoter. The CMVm promoter encompassed the sequence from +75 to −53 of the full CMV promoter. A fragment containing a sequence encoding EGFP was derived from pEGFP-C1 (bp positions 580-1665, Clontech, Palo Alto, Calif.) and inserted into pAcUW21 (PharMingen International). The p10 promoter in this plasmid was replaced by either the CMVm or the heat shock 70 promoter. The resulting plasmids were named pAPcmE and pAPhE, respectively. The same EGFP-encoding sequence was inserted into pBacPAK8 (Clontech, Palo Alto, Calif.) and under the control of the polyhedrin promoter. This resulting plasmid was named pABpE.

Generation of Recombinant Baculoviruses

To generate recombinant viruses, the just-described plasmids were cotransfected with linearized viral DNA into Sf21 cells respectively. More specifically, Sf21 cells, derived from Spodoptera frugiperda, were maintained as monolayer cultures in tissue culture flasks at 28° C. in a TNM-FH medium supplemented with 8% heat-inactivated fetal bovine serum. pAPcmE or pAPhE were cotransfected with linearized vAcRP23.LacZ (PharMingen, International), and pABpE was cotransfected with linearized BaculoGold (PharMingen, International). All transfections were conducted using Lipofectin (Invitrogen, Carlsbad, Calif.) according to the manufacturer's protocol. After three rounds of standard purification, pure recombinant viruses were isolated. The recombinant viruses thus obtained were named vAPcmE, vAPhE, and vABpE, respectively. All viral stocks were prepared and the corresponding titers determined according to standard protocols (O'reilly et al., Baculovirus Expression Vectors: A Laboratory Manual; Oxford University Press: New York, 1994).

Nucleic Acid Extraction by Column

Viral DNA was extracted by column using a viral nucleic acid isolation kit (High Pure, Roche Mannheim, Germany) according to the manufacturer's protocol. Poly(A) carrier RNA provided in the kit was added to a binding buffer (6 M guanidine-HCl, 10 mM urea, 10 mM Tris-HCl, 20% Triton X-100 (v/v), pH 4.4) to form a working solution. Then, 200 μL of the working solution and 0.9 mg proteinase K were added to 200 μL of virus stock and incubated at 72° C for 10 minutes. Then, the resultant sample was mixed with 100 μL of 2-propanol. The mixture was loaded into a High Pure filter tube and centrifuged for 1 minute at 9000 rpm in a standard tabletop centrifuge. The filter tube was removed and put into a clean collection tube, and the flowthrough was discarded. Next, 500 μL of an inhibitor removal buffer (5 M guanidine-HCl, 20 mM Tris-HCl, pH 6.6) was added to the upper reservoir of the filter tube and centrifuged for 1 minute at 9000 rpm. A wash buffer (450 μL, containing 20 mM NaCl and 2 mM Tris-HCl, pH 7.5) was then added to the upper reservoir of the filter tube and centrifuged for 1 minute at 9000 rpm, followed by 10 seconds at 13,000 rpm, to remove residual wash buffer. The collection tube was discarded, and a nuclease-free, sterilized 1.5-mL eppendorf tube was inserted. Fifty microliters of double-distilled water was added to the upper reservoir of the filter tube and allowed to stand for 2 minutes at room temperature, and then the tube was centrifuged for 1 minute at 9000 rpm.

Nucleic Acid Extraction by Phenol/Chloroform

Viral DNA was extracted using phenol/chloroform method from 200 μL of virus stock medium with a titer 4.3×10⁸ PFU/mL. The solution was centrifuged at 100,000 g at 4° C. for 45 minutes to pellet the budded viruses. Next, 1 mL of a virus disruption buffer (10 mM Tris-HCl, pH 7.6; 10 mM EDTA, 0.25% SDS) was added, and the pellet was carefully resuspended by pipeting up and down gently using a pipet with a cutoff tip. Proteinase K was then added to a final concentration of 500 μg/mL to digest viral proteins at 37° C. with gently mixing for 4 hours. Then DNA was purified by sequential phenol, phenol-chloroform, and chloroform extractions. A 2.5 volume of 100% ethanol and 0.1 volume of 3 M NaOAc, pH 5.2, was added to precipitate the viral DNA at −20° C. for 1 hour. After centrifugation, the viral DNA was washed with 70% ethanol and then dried by vacuum for 5 minutes. The viral DNA was then resuspended with a 1×tris-EDTA buffer.

### EXAMPLE 1

In this example, the specificity and sensitivity of a method of this invention was examined and compared with those of conventional titer determination methods. A region from the immediate-early IE-1 gene (Chisholm et al., J Virol. 1988 September; 62(9):3193-200) was used.

A stock of wild-type AcMNPV (5×10⁸ pfu/mL) was 10-fold diluted. DNAs were purified from each of these diluted viral solutions by the above-described column extraction and phenol/chloroform extraction methods. The quality of DNAs by these two methods were compared using DNA slot blot analysis according to the method described in Lin et al., J. Virol. 1999, 73, 128-139. Briefly, viral DNA was extracted from 200 μL of medium of infected cells. The DNA was denatured in 0.4 N NaOH and 10 mM EDTA at 100° C. for 10 minutes. The denatured DNA was then transferred to a Zeta-Probe membrane using MilliBlot (Millipore, USA) according to the manufacturer's instructions. After UVcross-linking, Dig-labeled ie-1 was used as a probe to hybridize the immobilized viral DNA. It was found that the phenol/chloroform extraction method was lengthier and more complicated than the column extraction method. Viral DNA purified from the phenol/chloroform extraction method was not good enough to perform Q-PCR quantification. In contrast, viral DNA prepared by column was consistently good enough to perform Q-PCR.

For Q-PCR, primers used for detecting viral-associated DNA were chosen from the open reading frame of a baculovirus essential gene ie-1 (Chisholm et al., J. Virol. 1988, 62, 3193-3200). The sequences of the ie-1-specific primers were as follows: forward primer 5′-CCCGTAACGGACCTCGTACTT and reverse primer 5′-TTATCGAGATTTATTTGCATACAACAAG. For the Q-PCR, a 10 μL aliquot of extracted nucleic acid was used for real-time amplification in a final volume of 20 μL reaction mixtures, which contained 1×SYBR Green I master mix (Roche Mannheim, Germany), MgCl₂ (4 mM), and 0.5 μM of the forward and reverse primers. A negative control was included. It contained only 10 μL of reaction mixture and 10 μL of water. The Q-PCR titration assay was performed in a LightCycler instrument (Roche Mannheim, Germany). The LightCycler provided rapid (30- to 40-minute) automation of PCR using capillary cuvettes. The PCR reagents and specimen extracts were centrifuged in capillary tubes to facilitate mixing. The capillary tubes were placed in the LightCycler. After all capillaries were sealed, DNA amplification was conducted using the following protocol: 95° C. for 30 seconds for one cycle, followed by denaturation at 95° C. for 30 seconds, annealing at 63° C. for 5 seconds, and primer extension at 72° C. for 10 seconds. In each PCR cycle, the amplified target nucleic acids bound to SYBR Green I. The fluorescence signal was monitored at 80° C. for 45 cycles. Based on the change in the fluorescence intensity, the cross point for each diluted sample was measured.

Meanwhile, the titer of each diluted viral solution was determined, by the end-point dilution method known in the art, based on the formation of occlusion bodies. The titers determined were plotted against the corresponding cross points. The results showed that the plotting of corresponding cross points and determined titers had a precise linear correlation (R²=0.9982). The function for this linear correlation was found to be as follows:
y=−1.2632×Ln(x)+32.83 (Formula I)

In the formula, “y” stands for a cross point value (cycle number) and “x” stands for a baculovirus titer value (pfu/ml).

Following the above-described PCR amplification, a melting curve analysis was performed by gradually increasing the temperature (0.1° C./s) up to 95° C. Change in the fluorescence signal was continuously monitored. With the aid of the LightCycler software, the rate of fluorescence changes as a function of temperature was plotted. The melting curve of the PCR products from each viral solution was obtained and analyzed to confirm that the signals recorded were due to the amplification of the ie-1 products. It was found that for each sample, the major peak centered at 86° C., indication the amplified products were ie-1 specific.

As mentioned above, fluorescence intensity increased during the PCR due to the proportional binding of SYBR Green I to the PCR products. The changes in the intensity were measured and plotted as a function of cross points. It was found that in a range from 0.005 ng to 500 ng, (1) the PCR resulted in detectable a fluorescence signal and (2) the number of viral genomic DNA (i.e, amplicons) negatively correlated with the PCR cycle number. The log concentrations of the template were also plotted against the cross points. It was found that plot was linear in the range from 0.005 ng to 500 ng. These results suggest that the linear range for quantification of viral DNA is between 0.005 ng and 500 ng and that the plots are reliable and can be used to quantify virus in a wide range.

### EXAMPLE 2

In Example 1 above, observations of occlusion body formation were relied on to determine titers of baculovirus successfully. Nonetheless, formation of occlusion body is very slow and obscure. In this example, an end-point dilution method based on EGFP fluorescence was used to achieve more precise and reproducible viral titers to calibrate the titers estimated by Q-PCR.

More specifically, a solution of vAPhE with a known titer was 10-fold serially diluted. The viral DNA of in each diluted sample was purified, and the corresponding cross point was determined by Q-PCR using the ie-1 primers in the same manner described in Example 1 above. Titers of the same 10-fold-diluted samples were also determined by end-point dilution based on EGFP fluorescence (Chao et al., Nature 1996, 380, 396-397 and Wilson, et al., Biotechniques 1997, 22, 674-678.). Data from 10 sets of Q-PCR and end-point dilutions were used to plot a standard curve. Based on this standard curve, a formula for virus titers against the cross points was obtained (R²=0.9971):
y=−1.4293×Ln(x)+38.965 (Formula II)

In the formula, “y” stands for a cross point value (cycle number) and “x” stands for a baculovirus titer value (pfu/ml).

This formula was then tested using vABpE and vAPcmE, the genomes of which contain the egfp gene. The initial virus titers for vABpE and vAPcmE were 1×10⁸, and 5×10⁸. After a series of 10-fold dilutions, the titer of each dilution was measured either by the end-point dilution method based on detection of green fluorescence or by Q-PCR method based on the just-described formula. The actual titers were plotted against the titers obtained by the two methods, respectively. It was found that the two plots for each virus were very similar, suggesting that the Q-PCR-based titer determination was both precise and reproducible.

### EXAMPLE 3

In this example, the above-described method was used to determine the titer of baculovirus in a sample. The virus vABpE, with an initial titer of 1×10⁸, was diluted on a 96-well plate to an end point at which each well should contain only one virus on average. The diluted viruses were allowed to propagate for 5 days before the supernatants were harvested from 10 randomly selected wells. This was to mimic the routine isolation of single viral clones used in the baculovirus expression vector system, and the viruses in each well were referred to as an individual clone. Usually, the titer of isolated viral clones should be determined before proceeding to any further applications. To test whether the above-described formula could be applied to viruses with unknown titers, the titers of the virus solutions from these 10 wells were examined by either the end-point dilution assay, based on green fluorescence, or Q-PCR based on the ie-1 primer. Titer variation between these two methods was compared using the two-tailed F-test. The results were summarized in Table 1 below:

TABLE 1
Titers Determination by End-Point Dilution and Q-PCR

| | Eend-point | | |
|---|---|---|---|
| | dilution^{a} | Q-PCR^{b}(×10⁸ | Cross point |
| Clone | (×10⁸ pfu/mL) | pfu/mL) | (cycle number) |
| clone 1 | 6.91 | 2.60 | 11.29 |
| clone 2 | 2.14 | 1.31 | 12.26 |
| clone 3 | 2.08 | 1.47 | 12.11 |
| clone 4 | 2.34 | 1.05 | 12.42 |
| clone 5 | 7.12 | 2.25 | 11.60 |
| clone 6 | 6.51 | 2.81 | 11.22 |
| clone 7 | 4.18 | 1.40 | 12.12 |
| clone 8 | 2.68 | 0.92 | 12.66 |
| clone 9 | 3.58 | 1.32 | 12.24 |
| clone 10 | 0.42 | 0.49 | 13.69 |
| Mean | 3.80 | 2.16 | |
| Standard deviation | 2.33 | 1.81 | |
| Standard error | 3 | 3 | |

| | |
|---|---|
| F-test | 1.657^{c} |
| Student's t test | 1.96^{c} |
| confidence (95%) | 3.8 × 10⁸ 1 ± .66 × 10⁸ 2.16 × 10⁸ ± 1.29 × 10⁸ |

^{a}determined by the emission of green fluorescence.
^{b}determined by ie-1 primer.
^{c}level of significance, 5%.

As shown in Table 1, no significant difference was found in these two methods. Also, no significant difference was shown between the means of the titers obtained by these two methods using the two-tailed Student's t-test for paired samples. These results indicate (i) that titers determined by the Q-PCR-based method correlate well with those obtained by the conventional end-point dilution (ii) that the end-point dilution method could be replaced by the Q-PCR-based assay.

### EXAMPLE 4

In this example, the above-described end-point dilution method and Q-PCR-based method were used to determine titers of virus stocks that had been stored at 4° C. for 6 months, 1 year, 2 years, 3 years, or 4 years. Before storing, all samples were quantified by the end-point dilution method. The titers determined before storing (“End-point dilution (original)”) and the titer determined after storing (“End-point dilution (current)” and “Q-PCR (current)”) were summarized in Table 2 below.

TABLE 2
Titers of Virus Stocks after Storage

| | Duration of storage^{a} | | | | |
|---|---|---|---|---|---|
| Quantification methods | 6 months | 1 year | 2 years | 3 years | 4 years |
| End-point dilution (original)^{b} | 2.57 × 10⁸ | 8.5 × 10⁸ | 3.81 × 10⁸ | 9.4 × 10⁸ | 1.26 × 10⁸ |
| End-point dilution (current)^{c} | 6.9 × 10⁸ | 8.9 × 10⁸ | 2.5 × 10⁸ | 6.9 × 10⁸ | 1.0 × 10⁸ |
| Q-PCR (current) | 4.3 × 10⁸ | 6.3 × 10⁸ | 3.99 × 10⁸ | 2.3 × 10⁸ | 1.8 × 10⁸ |

^{a}After purification, virus stocks were stored at 4° C. for various time as indicated.
^{b}These were titers estimated individually at the beginning of virus stock.
^{c}These were current estimation of individually virus titers of the stocks.

As shown in Table 1, the titers were similar regardless of the quantification methods used. More interestingly, the titers had not changed from the originally measured titers even after 4 years of storage. The PCR products from the capillary cuvettes were collected and analyzed by 3% agarose gel. The results proved that the 150-bp amplicons were the only product amplified by the Q-PCR experiments.

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the scope of the following claims.

1. A pair of primers for amplifying a nucleic acid of baculovirus, each containing an oligo-nucleotide selected from the baculovirus ie-1gene region and being 5-100 nucleotides in length.

2. The pair of primers of claim 1, wherein each primer is 10-50 nucleotides in length.

3. The pair of primers of claim 1, wherein the oligo-nucleotides in the pair of primers are SEQ ID NOs: 1 and 2, respectively.

4. The pair of primers of claim 3, wherein each primer is 10-50 nucleotides in length.

5. A nucleic acid obtained from amplification of a baculovirus nucleic acid template with a pair of primers, each containing an oligo-nucleotide selected from the baculovirus ie-1gene region, wherein each primer is 5-100 nucleotides in length.

6. The nucleic acid of claim 5, wherein the nucleic acid is 50- 2,000 nucleotides in length.

7. The nucleic acid of claim 6, wherein the nucleic acid is 100-1,000 nucleotides in length.

8. The nucleic acid of claim 5, wherein the oligo-nucleotides in the pair of primers are SEQ ID NOs: 1 and 2, respectively.

9. The nucleic acid of claim 8, wherein the nucleic acid is 50-2,000 nucleotides in length.

10. The nucleic acid of claim 9, wherein the nucleic acid is 10-1,000 nucleotides in length.

11. The nucleic acid of claim 10, wherein the nucleic acid contains SEQ ID NO: 3 or the complement thereof.

12. A method for quantifying a microorganism in a sample, comprising:
amplifying, by a polymerase chain reaction, a nucleic acid in the genome of the microorganism to form a double-stranded nucleic acid product;
contacting the nucleic acid product with a label in a solution for binding therebetween; and
quantifying the microorganism by monitoring a signal produced by the bound label, the intensity of the signal being a function of the quantity of the microorganism in the sample.

13. The method of claim 12, wherein the label is a fluorophore.

14. The method of claim 13, wherein the fluorophore is SYBR Green I.

15. The method of claim 12, further comprising, after the contacting step, heating the solution to a temperature that is above the melting temperature of the double-stranded nucleic acid product, and confirming the identity of the microorganism by monitoring a change in the intensity of the signal produced by the label as the temperature rises.

16. The method of claim 12, wherein the microorganism is quantified based on Formula I.

17. The method of claim 12, wherein the microorganism is quantified based on Formula II.

18. The method of claim 12, wherein the microorganism is a virus.

19. The method of claim 18, wherein the virus is a baculovirus.

20. The method of claim 19, wherein the baculovirus is a recombinant baculovirus and the nucleic acid in the genome is heterologous.

21. The method of claim 20, wherein the nucleic acid encodes GFP or Luciferase, or a fragment thereof.

22. The method of claim 21, wherein the polymerase chain reaction is conducted with a pair of primers that contain, SEQ ID NOs: 4 and 5, respectively, or contain SEQ ID NOs: 6 and 7, respectively.

23. The method of claim 19, wherein the nucleic acid in the baculovirus genome is selected from the ie-1 gene region of the baculovirus.

24. The method of claim 23, wherein the label is a fluorophore.

25. The method of claim 24, wherein the fluorophore is SYBR Green I.

26. The method of claim 23, further comprising, after the contacting step, heating the solution to a temperature that is above the melting temperature of the double-stranded nucleic acid product, and confirming the identity of the microorganism by monitoring a change in the intensity of the signal produced by the label as the temperature rises.

27. The method of claim 23, wherein the microorganism is quantified based on Formula I.

28. The method of claim 27, wherein the microorganism is quantified based on Formula II.

29. The method of claim 23, wherein the polymerase chain reaction is conducted with a pair of primer that contain SEQ ID NOs: 1 and 2, respectively.

30. The method of claim 29, wherein the label is a fluorophore.

31. The method of claim 30, wherein the fluorophore is SYBR Green I.

32. The method of claim 31, further comprising, after the contacting step, heating the solution to a temperature that is above the melting temperature of the double-stranded nucleic acid product, and confirming the identity of the microorganism by monitoring a change in the intensity of the signal produced by the label as the temperature rises.

33. The method of claim 32, wherein the microorganism is quantified based on Formula I.

34. The method of claim 33, wherein the microorganism is quantified based on Formula II.


SEQUENCE LISTING (8 sequences)

SEQ ID NO: 1 - DNA, 21 bp, Artificial Sequence
  Primer
cccgtaacggacctcgtactt

SEQ ID NO: 2 - DNA, 28 bp, Artificial Sequence
  Primer
ttatcgagatttatttgcatacaacaag

SEQ ID NO: 3 - DNA, 141 bp, Artificial Sequence
  Synthetically generated oligonucleotide
cccgtaacggacctcgtacttttggcttcaaaggttttgcgcacagacaaaatgtgccacacttgcagctctgcatgtgtgcgcgttaccacaaatcccaacggcgcagtgtacttgttgtatgcaaataaatctcgataa

SEQ ID NO: 4 - DNA, 25 bp, Artificial Sequence
  Primer
caacagccacaacgtctatatcatg

SEQ ID NO: 5 - DNA, 21 bp, Artificial Sequence
  Primer
atgttgtggcggatcttgaag

SEQ ID NO: 6 - DNA, 20 bp, Artificial Sequence
  Primer
cggcgccattctatcctcta

SEQ ID NO: 7 - DNA, 23 bp, Artificial Sequence
  Primer
accagggcgtatctcttcatagc

SEQ ID NO: 8 - DNA, 2655 bp, Autographa californica nucleopolyhedrovirus
  Primer
atcgatgtctttgtgatgcgcgcgacatttttgtaggttattgataaaatgaacggatacgttgcccgacattatcattaaatccttggcgtagaatttgtcgggtccattgtccgtgtgcgctagcatgcccgtaacggacctcgtacttttggcttcaaaggttttgcgcacagacaaaatgtgccacacttgcagctctgcatgtgtgcgcgttaccacaaatcccaacggcgcagtgtacttgttgtatgcaaataaatctcgataaaggcgcggcgcgcgaatgcagctgatcacgtacgctcctcgtgttccgttcaaggacggtgttatcgacctcagattaatgtttatcggccgactgttttcgtatccgctcaccaaacgcgtttttgcattaacattgtatgtcggcggatgttctatatctaatttgaataaataaacgataaccgcgttggttttagagggcataataaaagaaatattgttatcgtgttcgccattagggcagtataaattgacgttcatgttggatattgtttcagttgcaagttgacactggcggcgacaagatcgtgaacaaccaagtgactatgacgcaaattaattttaacgcgtcgtacaccagcgcttcgacgccgtcccgagcgtcgttcgacaacagctattcagagttttgtgataaacaacccaacgactatttaagttattataaccatcccaccccggatggagccgacacggtgatatctgacagcgagactgcggcagcttcaaactttttggcaagcgtcaactcgttaactgataatgatttagtggaatgtttgctcaagaccactgataatctcgaagaagcagttagttctgcttattattcggaatcccttgagcagcctgttgtggagcaaccatcgcccagttctgcttatcatgcggaatcttttgagcattctgctggtgtgaaccaaccatcggcaactggaactaaacggaagctggacgaatacttggacaattcacaaggtgtggtgggccagtttaacaaaattaaattgaggcctaaatacaagaaaagcacaattcaaagctgtgcaacccttgaacagacaattaatcacaacacgaacatttgcacggtcgcttcaactcaagaaattacgcattattttactaatgattttgcgccgtatttaatgcgtttcgacgacaacgactacaattccaacaggttctccgaccatatgtccgaaactggttattacatgtttgtggttaaaaaaagtgaagtgaagccgtttgaaattatatttgccaagtacgtgagcaatgtggtttacgaatatacaaacaattattacatggtagataatcgcgtgtttgtggtaacttttgataaaattaggtttatgatttcgtacaatttggttaaagaaaccggcatagaaattcctcattctcaagatgtgtgcaacggcgagacggctgcacaaaattgtaaaaaatgccatttcgtcgatgtgcaccacacgtttaaagctgctctgacttcatattttaatttagatatgtattacgcgcaaaccacatttgtgactttgttacaatcgttgggcgaaagaaaatgtgggtttcttttgagcaagttgtacgaaatgtatcaagataaaaatttatttactttgcctattatgcttagtcgtaaagagagtaatgaaattgagactgcatctaataatttctttgtatcgccgtatgtgagtcaaatattaaagtattcggaaagtgtgcagtttcccgacaatcccccaaacaaatatgtggtggacaatttaaatttaattgttaacaaaaaaagtacgctcacgtacaaatacagcagcgtcgctaatcttttgtttaataattataaatatcatgacaatattgcgagtaataataacgcagaaaatttaaaaaaggttaagaaggaggacggcagcatgcacattgtcgaacagtatttgactcagaatgtagataatgtaaagggtcacaattttatagtattgtctttcaaaaacgaggagcgattgactatagctaagaaaaacaaagagttttattggatttctggcgaaattaaagatgtagacgttagtcaagtaattcaaaaatataatagatttaagcatcacatgtttgtaatcggtaaagtgaaccgaagagagagcactacattgcacaataatttgttaaaattgttagctttaatattacagggtctggttccgttgtccgacgctataacgtttgcggaacaaaaactaaattgtaaatataaaaaattcgaatttaattaattatacatatattttgaatttaattaattatacatatattttatattatttttgtcttttattatcgaggggccgttgttggtgtggggttttgcatagaaataacaatgggagttggcgacgttgctgcgccaacaccacctcctcctcctcctttcatcatgtatctgtagataaaataaaatattaaacctaaaaacaagaccgcgcctatcaacaaaatgataggcattaacttgccgcgacgctgtcactaacgttggacgatttgccgactaaaccttcatcgcccagtaaccaatctaga